(19) United States
(12) Patent Application Publication
(10) Pub. No.: US 2018/0064467 A1
(43) Pub. Date: Mar. 8, 2018

(54) IMPLANT AND FASTENER FIXATION DEVICES

(71) Applicant: COLLAB, LLC, Whitmore Lake, MI (US)

(72) Inventor: Paul A. Zwirkoski, Hamburg Township, MI (US)

(21) Appl. No.: 15/489,320

(22) Filed: Apr. 17, 2017

Related U.S. Application Data
(63) Continuation of application No. 14/234,194, filed on Apr. 16, 2014, now abandoned.
(60) Provisional application No. 61/518,611, filed on May 9, 2011.

(51) Int. Cl. A61B 17/68; A61B 17/86; A61B 17/88; A61B 17/84

(52) U.S. Cl. CPC A61B 17/686; A61B 17/86; A61B 2017/8655; A61B 17/8875; A61B 17/8695; A61B 17/869; A61B 17/8685; A61B 17/864; A61B 17/846

(57) ABSTRACT

Described here are delivery or fastener device fixation implant devices, delivery instrumentation, and methods for using them. In particular, the description relates to implants having a having a strength sufficient to support, anchor, secure, maintain, or to otherwise repair an elongated delivery or fastener device anywhere such a device, such as, for example, a bone screw, may need further securing within a substrate it is inserted into, such as, for example, within human or animal tissue, such as bone tissue.

### REFERENCE TO RELATED APPLICATIONS

This application is a Continuation of U.S. application Ser. No. 14/234,194, which was the US National Phase under 35 USC §371 of PCT/US2012/000239 having an international filing date of May 8, 2012, which is entitled to the benefit of the filing date of U.S. Provisional Application No. 61/518,611, filed May 8, 2011, the entirety of which is incorporated herein by reference.

### FIELD

Described here are delivery or fastener device fixation implant devices, delivery instrumentation, and methods for using them. In particular, the description relates to implants having a having a strength sufficient to support, anchor, secure, maintain, or to otherwise repair an elongated delivery or fastener device anywhere such a device, such as, for example, a bone screw, may need further securing within a substrate it is inserted into, such as, for example, within human or animal tissue, such as bone tissue. Also described are instrumentation devices for delivering fastener fixation devices into a desired location. Also, methods are described for inserting these implants into desired locations for repairing or anchoring fasteners, such as, for example, tissue fasteners such as bone screws.

### BACKGROUND

The present invention as disclosed herein provides implant devices, delivery instruments and methods for their use for securing delivery or fastener devices within a substrate or tissue that the device is inserted into, such as, for example, a bone screw inserted into cortical or cancellous bone. Currently, delivery or fastener devices, such as, for example, drainage tubes, delivery cannulas, screws, bolts or other devices are limited in their ability to provide long lasting load bearing capabilities due to a multitude of factors, such as, for example, the deterioration of substrate or bone tissue, that cause traditional delivery or fastener devices to loosen over time. Typically when these devices loosen they must be removed and replaced with a larger device to maintain the desired load bearing capabilities. It would be advantageous to provide a device that could provide anchoring, repairing and/or stabilization of a fastener device without replacing the delivery device or fastener. U.S. patent application Ser. No. 10/866,219, Ser. No. 11/298,961, and Ser. No. 12/616,843 by Zwirkoski disclose fixation devices comprised of flexibility connected segments having sufficient strength to anchor, support and/or repair delivery or fastener devices such as, for example, a bone screw. However, these patent applications do not disclose the novel embodiments and methods of use disclosed in the instant invention.

### BRIEF SUMMARY

Broadly, described here are various devices, such as guide rings, for securing fixation implants in precise desired locations delivery or fastener devices within a substrate cavity, non-flexibly connected segmented implants for securing fasteners within a cavity space, helical implants, instrumentation for the delivery of fastener fixation devices, and methods of using these devices to anchor, secure and/or repair a delivery device or fastener. Generally, fastener fixation implants, some of which are described herein and elsewhere, can be arranged and delivered into fastener space in a controlled and measured way through the use of structural implant guide rings or related implants. In addition, fastener fixation delivery devices are described that allow for the precise placement of the implants and guide rings.

Methods are described herein for using various guide components and instrumentation to achieve optimal placement and anchoring strength for fastener fixation implants.

### BRIEF DESCRIPTION OF THE DRAWINGS

Embodiments or variations are now described by way of example with reference to the accompanying drawing.

FIG. 1 shows an embodiment of an elongated fixation device surrounded by a fixation implant with a proximal and distal fixation implant guide ring.

FIG. 2A is a top view of a proximal fixation implant guide ring.

FIG. 2B is a perspective view of the proximal fixation implant guide ring of FIG. 2A.

FIG. 3A is a perspective view of a proximal fixation implant guide ring with a locking tab.

FIG. 3B is a perspective view of a proximal fixation implant guide ring with locking tabs.

FIG. 4A is a top view of a proximal fixation implant guide ring with a recessed locking component.

FIG. 4B is a front view of the proximal fixation implant guide ring with a recessed locking component of FIG. 4A.

FIG. 4C is a cross-sectional view of the proximal fixation implant guide ring with a recessed locking component of FIG. 4A.

FIG. 5A is a top plan view of an alternative embodiment of a proximal fixation implant guide ring with a recessed locking component.

FIG. 5B is a perspective view of the proximal fixation implant guide member with a recessed locking component of FIG. 5A.

FIG. 5C is a front view of the proximal fixation implant guide member with a recessed locking component of FIG. 5A.

FIG. 5D is a first cross-sectional view of the proximal fixation implant guide member with a recessed locking component of FIG. 5A.

FIG. 5E is a second cross-sectional view of the proximal fixation implant guide member with a recessed locking component of FIG. 5A.

FIG. 6A is a perspective view of an alternative proximal fixation implant guide ring.

FIG. 6B is a cross-sectional view of the proximal fixation implant guide ring of FIG. 6A with a fastener received therein.

FIG. 7A is a perspective view of a distal fixation implant guide ring.

FIG. 7B is a plan view of view of the distal fixation implant guide ring of FIG. 7A.

FIG. 7C is a cross-sectional view of the distal fixation implant guide member of FIG. 7B.

FIG. 8A is a perspective view of an alternative embodiment of a distal fixation implant guide ring.

FIG. 8B is a cross-sectional view of the distal fixation implant guide ring of FIG. 8A.

FIG. 9A is a top view of an alternative embodiment of an expandable helical designed fixation implant guide ring.

FIG. 9B is a perspective view of the expandable helical designed fixation implant guide ring of FIG. 9A.

FIG. 9C is a front view of the expandable helical designed fixation implant guide ring of FIG. 9A.

FIG. 9D is a cross-sectional view of the expandable helical designed fixation implant guide ring of FIG. 9A.

FIG. 10 shows a bone screw fastener with a proximal and distal fixation implant guide ring.

FIG. 11A is a top view of a fixation segmented implant attached to a distal fixation implant guide ring.

FIG. 11B is a front view of the fixation segmented implant attached to the distal fixation implant guide ring of FIG. 11A.

FIG. 12A is a front view of segmented fixation implants connected to distal and proximal fixation implant guide rings.

FIG. 12B is a perspective view of the segmented fixation implants connected to distal and proximal fixation implant guide rings of FIG. 12A.

FIG. 12C is an enlarged perspective view of the segmented fixation implants connected to distal and proximal fixation implant guide rings of FIG. 12A.

FIG. 13A is a front view of an alternative embodiment of segmented fixation implants with multiple fixation implant guide rings.

FIG. 13B is a perspective view of the segmented fixation implants with multiple fixation implant guide rings of FIG. 13A.

FIG. 14A is a top view of an alternative embodiment of segmented fixation implants with a partial fixation implant guide ring design.

FIG. 14B is a perspective view of the segmented fixation implants with partial fixation implant guide ring design of FIG. 14A.

FIG. 14C is a front view of the segmented fixation implants with partial fixation implant guide ring design of FIG. 14A.

FIG. 14D is a cross-sectional view of the segmented fixation implants with partial fixation implant guide ring design of FIG. 14A.

FIG. 15 shows a perspective of an alternative zigzagged embodiment of a segmented fixation implant.

FIG. 16 shows an alternative perspective of an alternative crisscrossed embodiment of a segmented fixation implant.

FIG. 17 shows a perspective of an alternative sinualsodial shaped segmented fixation implant embodiment.

FIG. 18 is a perspective of an alternative embodiment of a segmented fixation implant with overlapping sinualsodial shaped.

FIG. 19A is a front view of a helical shaped fixation implant.

FIG. 19B is a perspective view of the helical shaped fixation implant of FIG. 19A.

FIG. 20A is a front view of an alternative embodiment of a helical shaped fixation implant with a tighter wind than the helical shaped fixation implant of FIG. 19A.

FIG. 20B is a perspective view of the helical shaped fixation implant of FIG. 20A.

FIG. 21 shows a perspective of a helical shaped fixation implant on a bone screw.

FIG. 22 shows a helical shaped fixation device with the proximal and distal ends larger than the center.

FIG. 23 shows various perspectives of a guided fixation implant delivery device with slots.

FIG. 24 shows various perspectives of a guided fixation implant delivery device with proximal and distal guide rings.

FIG. 25 shows various perspectives of a guided fixation implant delivery cannula with an alternative embodiment of proximal and distal guide rings.

FIG. 26 is a delivery cannula with alternative implant guide rings.

FIG. 27A is a front view of a delivery cannula with an alternative implant guide ring.

FIG. 27B is a cross-sectional view of the delivery cannula of FIG. 27A, with the alternative implant guide ring received therein.

FIG. 28A is a front view of a fixation implant delivery driver.

FIG. 28B is an enlarged perspective view of the distal end of the fixation implant delivery driver of FIG. 28A.

FIG. 29A is a front view of an alternative fixation implant delivery driver.

FIG. 29B is an enlarged perspective view of the distal end of the fixation implant delivery driver of FIG. 29A.

FIG. 30A is a perspective view of a fixation implant delivery portal stabilizer.

FIG. 30B is a top view of the fixation implant delivery portal stabilizer of FIG. 30A.

FIG. 30C is a front view of the fixation implant delivery portal stabilizer of FIG. 30A.

FIG. 30D is a side view of the fixation implant delivery portal stabilizer of FIG. 30A.

FIG. 31A is a front view of guided fixation implant delivery components.

FIG. 31B is a side view of the guided fixation implant delivery components of FIG. 31A.

FIG. 32A is a cross-sectional view of fixation implant delivery device components.

FIG. 32B is a perspective view of fixation implant delivery device components.

FIG. 33 shows a delivery tamp.

FIG. 34A shows a first position in a sequence of positions of a guided fixation device and delivery of implants.

FIG. 34B shows a second position in a sequence of positions of the guided fixation device of FIG. 34A and delivery of implants.

FIG. 34C shows a third position in a sequence of positions of the guided fixation device of FIG. 34A and delivery of implants.

FIG. 34D is an enlarged view of implants following delivery via the guided fixation device of FIG. 34A.

### DETAILED DESCRIPTION

The fixation devices described herein are utilized with substrate delivery or fastener devices within a substrate, such as, for example, human tissue fastener devices such as, for example, a bone screw. The fixation implant support systems, delivery devices, and methods described herein are utilized with fixation implants for use with delivery devices or fasteners, particularly bone fasteners. While human tissue, such as bone is utilized as an example, any substrate could be utilized such as, for example wood, cement, drywall, or anywhere a delivery device or fastener needs fixation or securing.

Fixation Implant Guides

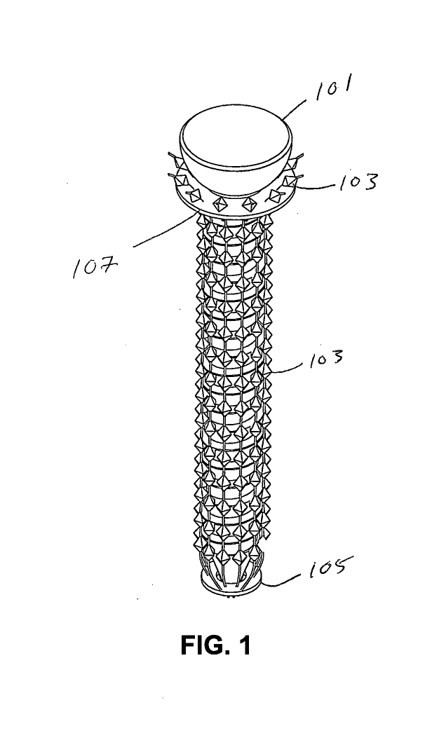

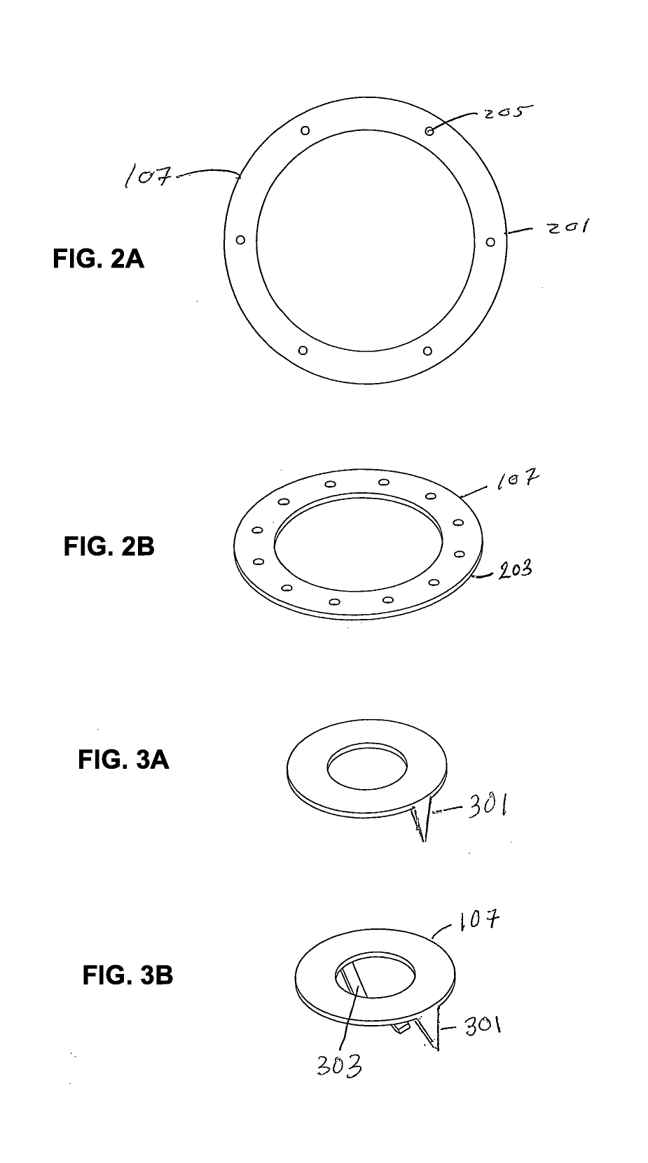

In one preferred embodiment as shown in FIG. 1 a fastener such as for example a bone screw 101 is encased with multiple flexibly connected segmented fastener fixation implants 103 that are placed around the fastener 101 with connections to a distal implant guide ring 105 and a proximal implant guide ring 107. These implant guide rings provide control and stability for the delivery and placement of the fastener fixation implants 103 in a desired location. FIGS. 2a-b show a top 201 and side 203 view of an example of a proximal implant guide ring 107 with openings 205 for placing the fastener fixation implants 103. In FIG. 2a six fastener fixation implant openings are utilized. Any number of fastener fixation implants 103 and corresponding openings 205 might be utilized however depending on the desired result.

FIGS. 3a-b show various perspectives of a proximal implant guide ring 107. FIG. 3a includes a penetrating locking tab 301 which can penetrate into the substrate, such as, for example, bone surrounding a delivery device or fastener such as a bone screw 101 and prevent rotation of the guide ring 107 once placed. One or more of the penetrating tabs 301 might be utilized to secure the guide ring. FIG. 3b shows a proximal implant guide ring with a penetrating locking tab 301 and a parallel flexible locking tab 303 that can be flexed into a position on a fastener 101 providing pressure on the fastener to prevent rotation of the proximal implant guide ring 107.

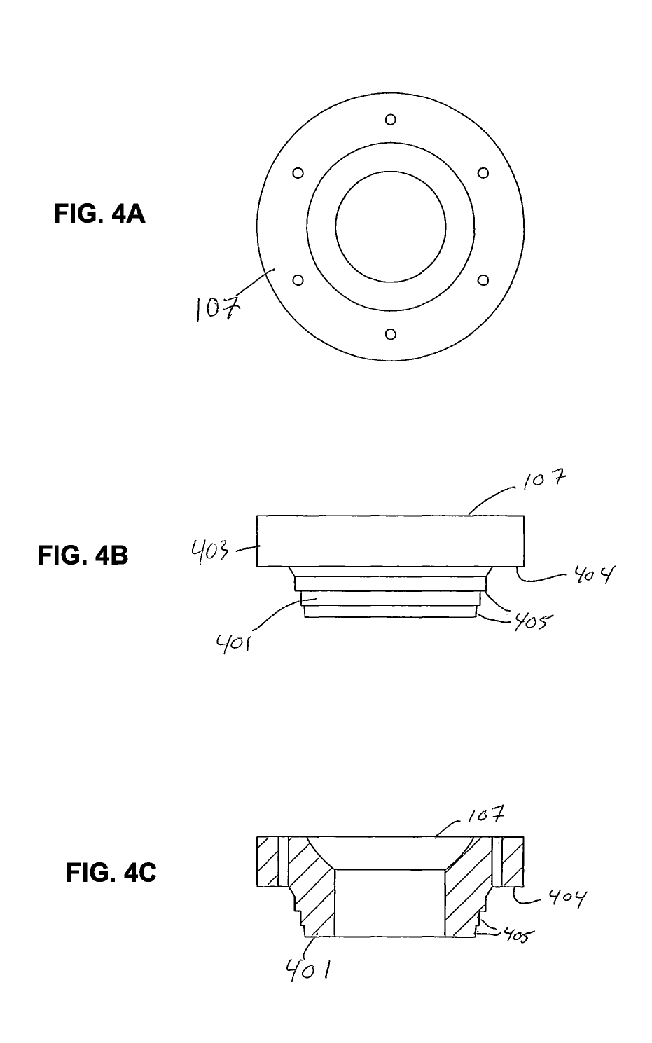

FIGS. 4a-c show a proximal implant guide ring 107 with a distal portion 401 that enters a fastener pathway into a substrate such as bone and positions the proximal implant guide 107 into the desired position without slippage. The proximal portion of the implant guide ring 403 might abut the proximal opening of the fastener opening 404. The distal portion might be layered and narrowed inwardly 405 at the distal portion 401 of the implant guide ring 107 as shown in FIGS. 4b-c.

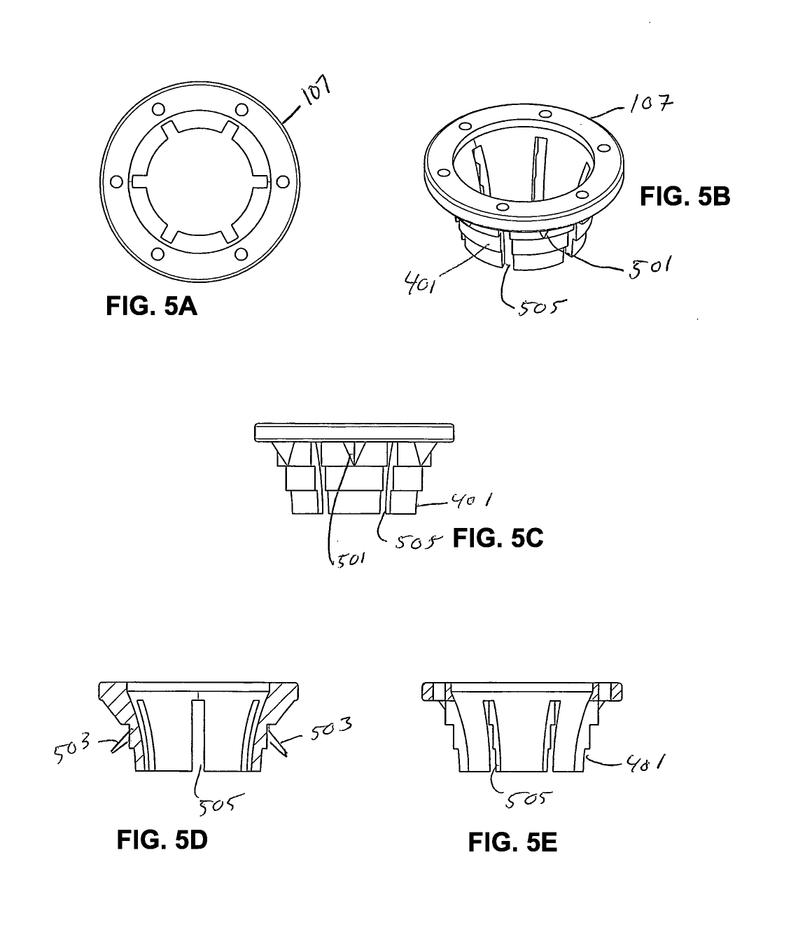

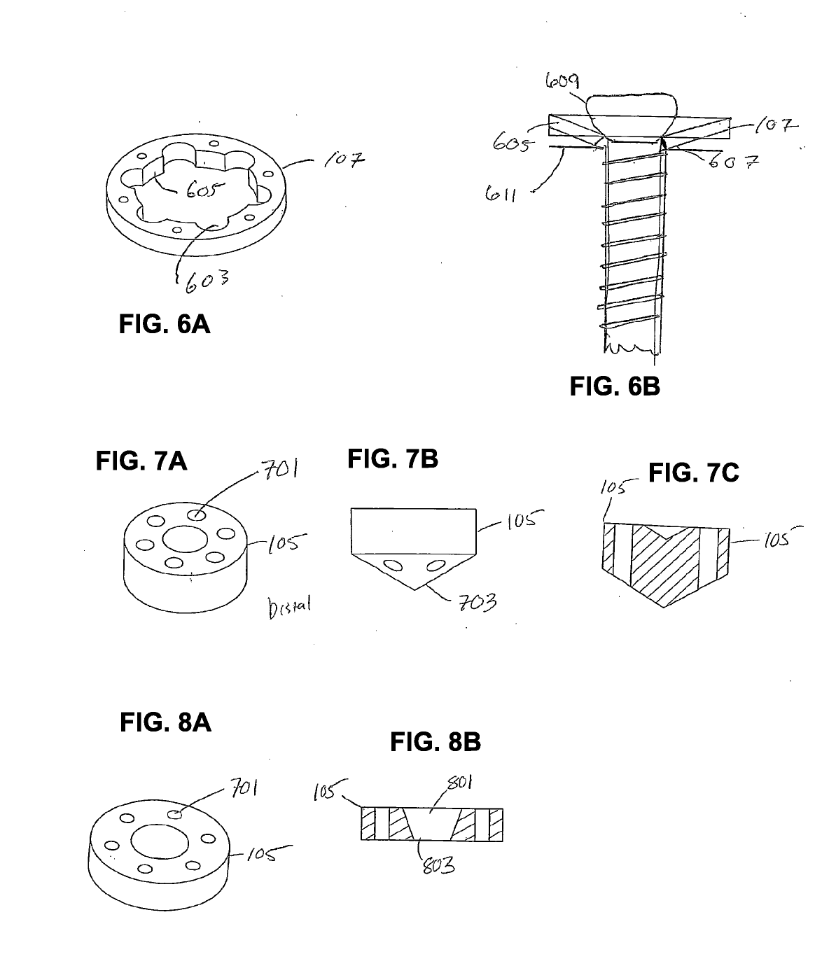

In one embodiment as shown in FIGS. 5a-e the distal sides of the proximal top of the proximal implant guide ring 107 might include gripping components such as scalloped spline teeth 501 that would help lock the guide 107 into the substrate such as bone surrounding the fastener opening. In one alternative embodiment the teeth might be located on the sides of the distal portion 503 of the proximal guide ring 401 as shown in FIG. 5d. While the preferred embodiment might include teeth and gripping component that might prevent rotation or movement of the proximal implant guide ring 107 any locking mechanism might be utilized. The distal portion 401 of the proximal implant guide ring 107 might contain spacings 505 that allow for the distal portion of the proximal guide ring 107 to be contracted and inserted into a fastener opening and upon placement expand out to secure the proximal guide ring 107. FIGS. 6a-b show an alternative shape for a proximal implant guide ring 107 whereby the inner wall 603 of the proximal guide ring 107 might have an irregular shape with flexible tabs 605 to assist with placement. In this embodiment the fastener 101 when placed into the guide ring would push the tabs 605 distally 607 between the proximal fastener head 609 and the substrate below the head 611, such as bone, providing a locking pressure on the proximal implant guide ring 107 preventing it from rotating or moving.

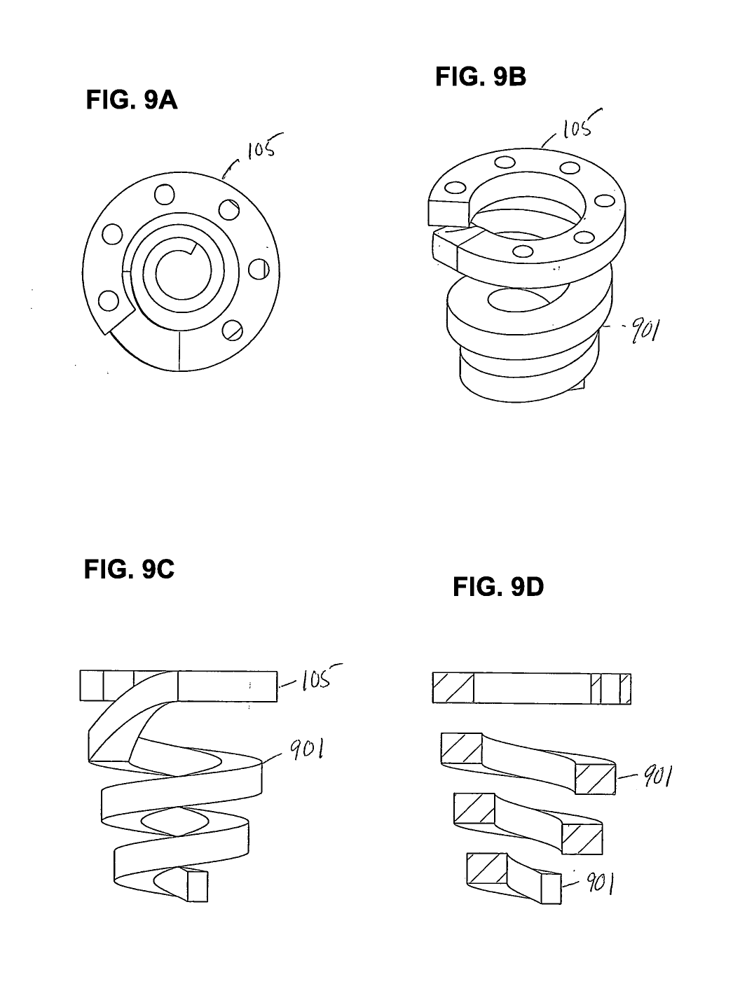

FIGS. 7a-c show various perspectives of one embodiment of a distal fixation implant guide ring 105. In this embodiment the fastener fixation implants 103 would be placed through the openings 701 allowing for desired placement of the fastener fixation implants along the length of the fastener 101. In one embodiment the distal end 703 of the distal guide ring 105 is tapered for placement at the bottom of the fastener 101 opening to provide self centering of the distal implant guide ring 105. FIGS. 8a-b show an alternative embodiment of a distal implant guide ring 105 without a self centering feature. In this embodiment the distal implant guide ring might include a tapered opening 801 that narrows distally 803 within the distal guide ring 105. FIGS. 9a-b show a helical shaped spring form expandable guide ring 901 of an implant guide ring 105 to facilitate securing the implant guide ring 105 and delivery device or fastener fixation implants 103. The distal end of the delivery device or fastener 101 pushes the distal implant guide ring 105 outwardly once placed.

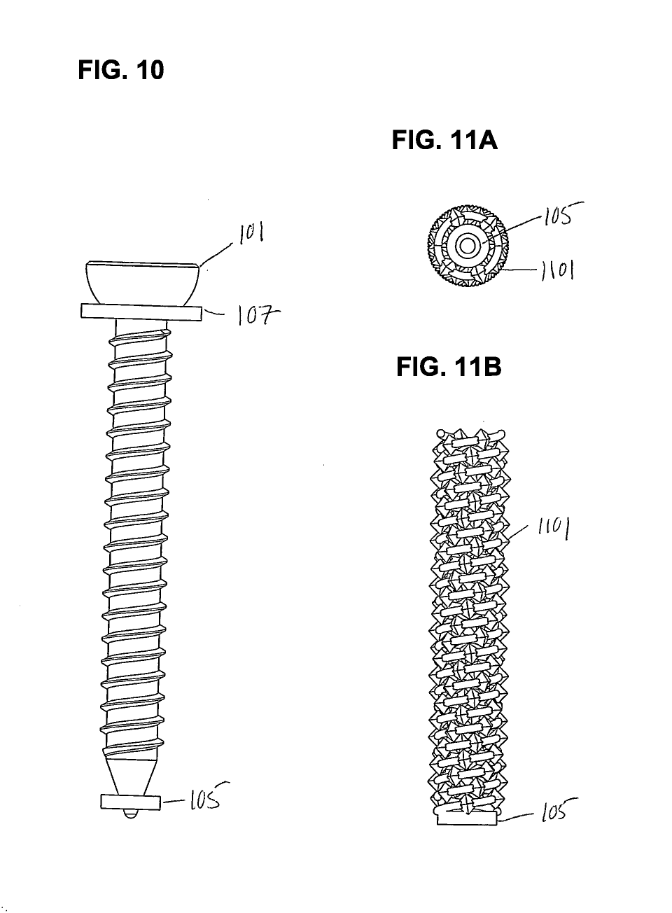

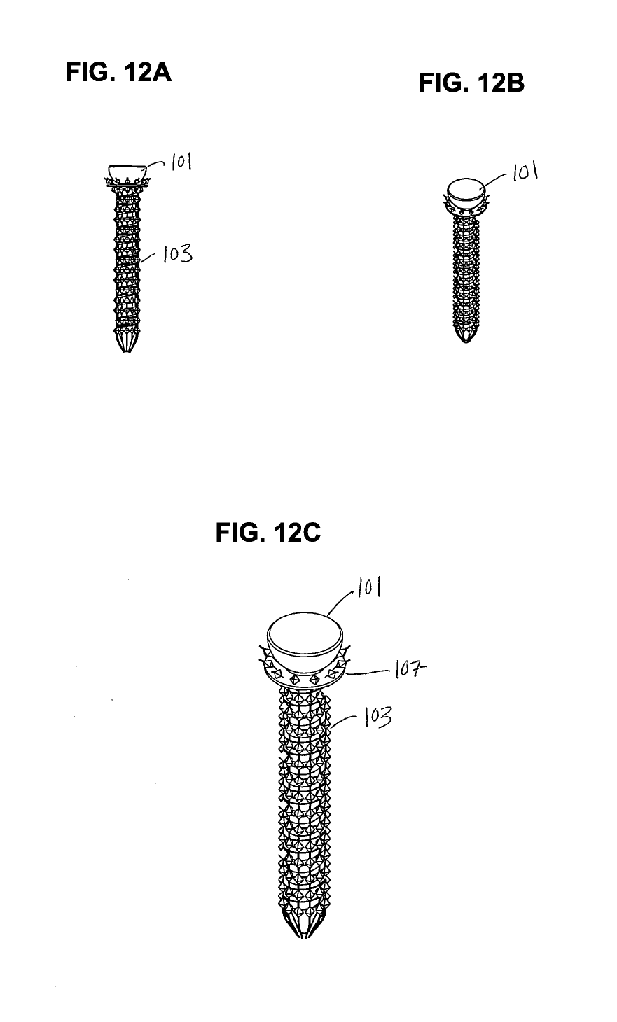

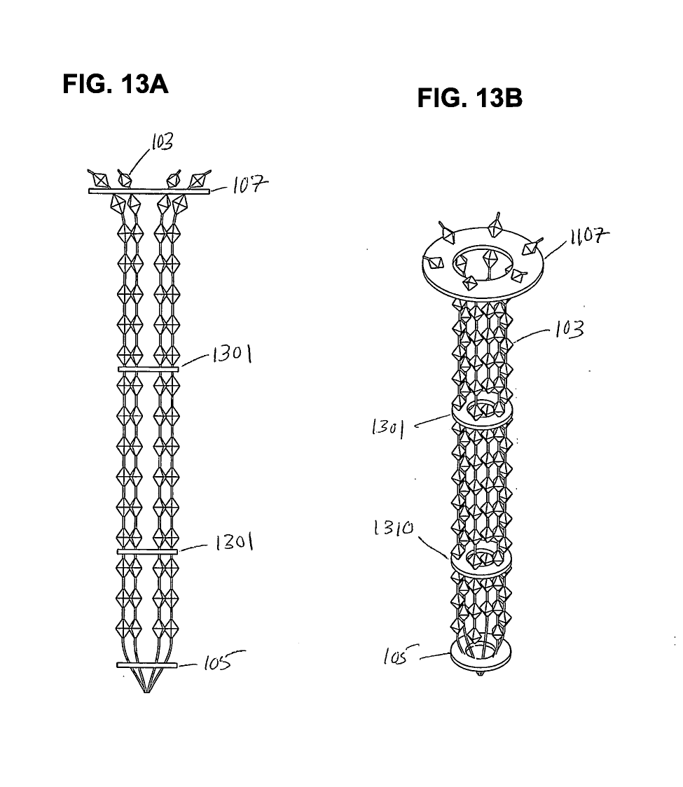

FIG. 10 shows a fastener 101 with a distal guide ring 105 and a proximal guide ring 107. FIGS. 11a-b show a helical segmented fixation implant 1101 connected to a distal implant guide ring 105. FIGS. 12a-c show a fixation implant 103 surrounding a fastener 101 and guided by a proximal implant guide ring 107 without a distal guide ring where the fixation implants 103 are not connected at all at their distal ends. FIGS. 13a-b show an alternative embodiment that includes three or more implant guide rings that include a distal ring 105, a proximal ring 107 and one or more implant guide rings 1301 located between the distal 105 and proximal implant guide rings 107 which might further stabilize and control the placement of the fixation implants 103. Any number of additional implant guide rings 1301 could be utilized.

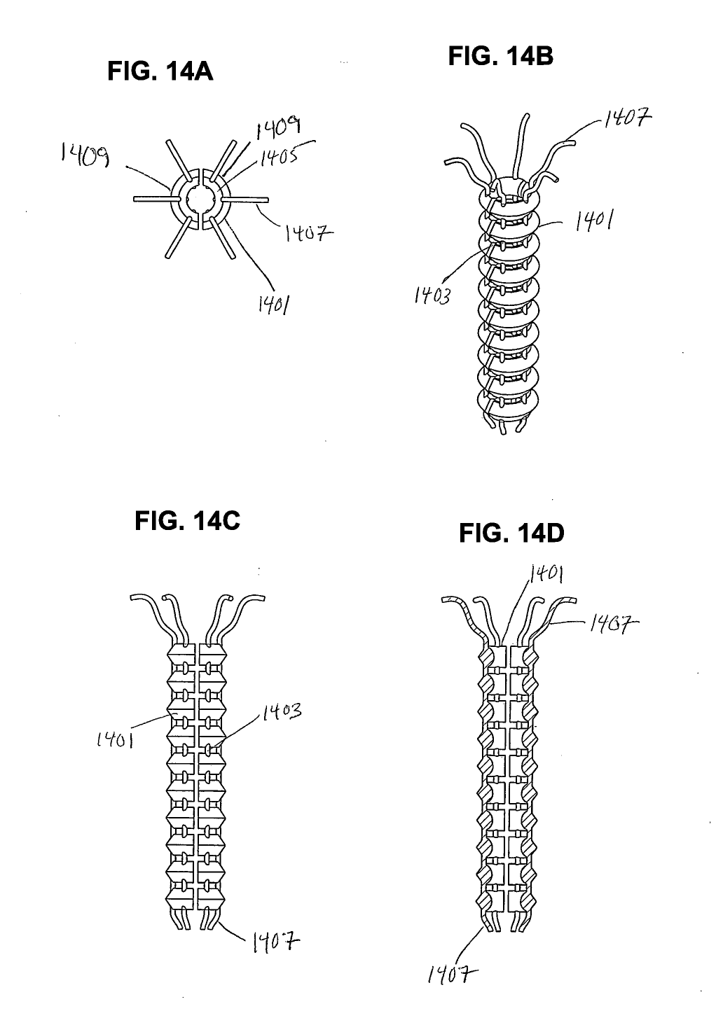

In FIGS. 14 a-c an alternative embodiment is shown where the segments 1401 of the fastener fixation implant 1403 are larger and act as partial implant guide rings that share two or more common connector strands 1407. In the disclosed embodiment the enlarged segment shapes 1401 are shown as two half circles 1409 with three connector strands 1407 passing through each segment. Any number of segments 1401 and/or connector strands 1407 could be utilized however that are able to achieve stabilization of the fastener fixation implants 103 around a fastener 101.

### Non-Flexible Connecting Fixation Implants

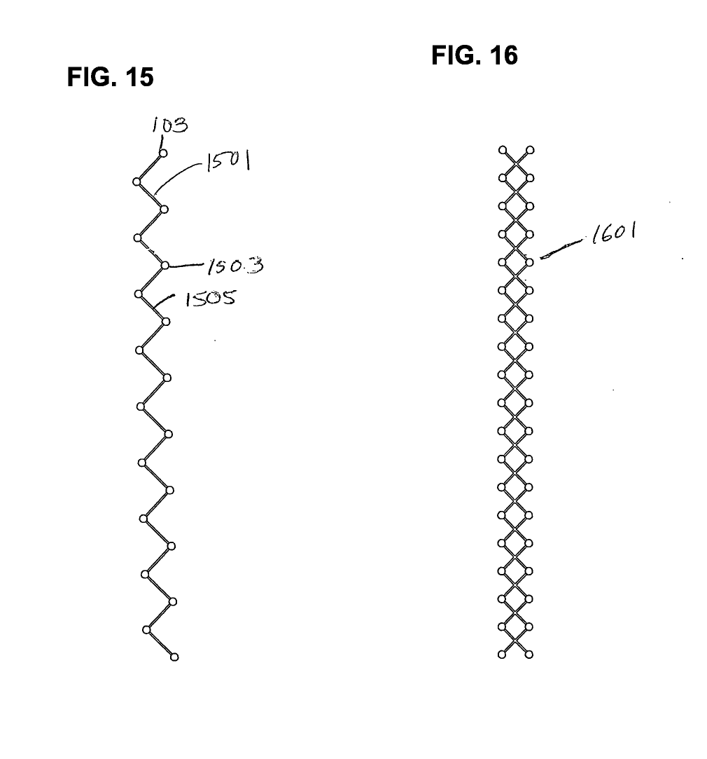

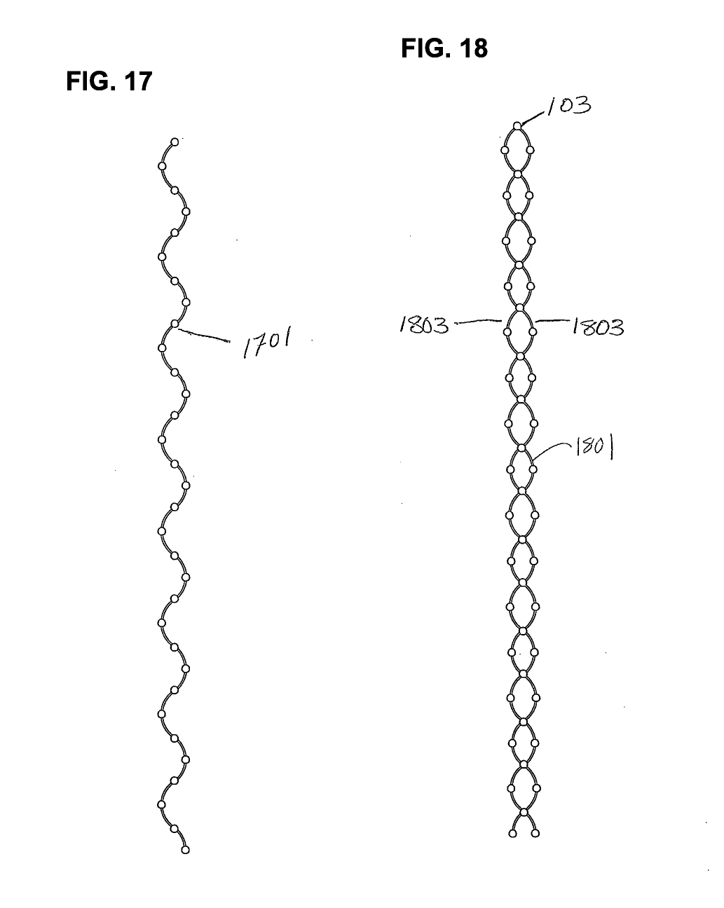

Prior disclosures of fastener fixation implant 103 designs disclose flexibly connected segments that allow for random placements of the implants. FIGS. 15-18 show example embodiments of non-flexibly connected segmented implants wherein the connecting materials are rigid and shaped to surround a delivery device or fastener so as to be fixated in position. FIG. 15 shows an alternative embodiment disclosing a zigzag fastener fixation implant design 1501. With this design the implant segments 1503 are connected by rigid connectors 1505 that form a back and forth zigzag shaped fastener fixation implant that would surround a fixation device 101. One or more implant strands 1505 might be utilized with each fastener 101 depending on the fixation desired. In one alternative two or more connector strands might by connected to form a crisscross fastener fixation implant 1601 as shown in FIG. 16. FIG. 17 discloses a sinualsodial wave form fastener fixation implant design 1701. FIG. 18 shows an implant design with two connected strands 1803 intertwined to allow for two or more sinualsodial wave forms 1801.

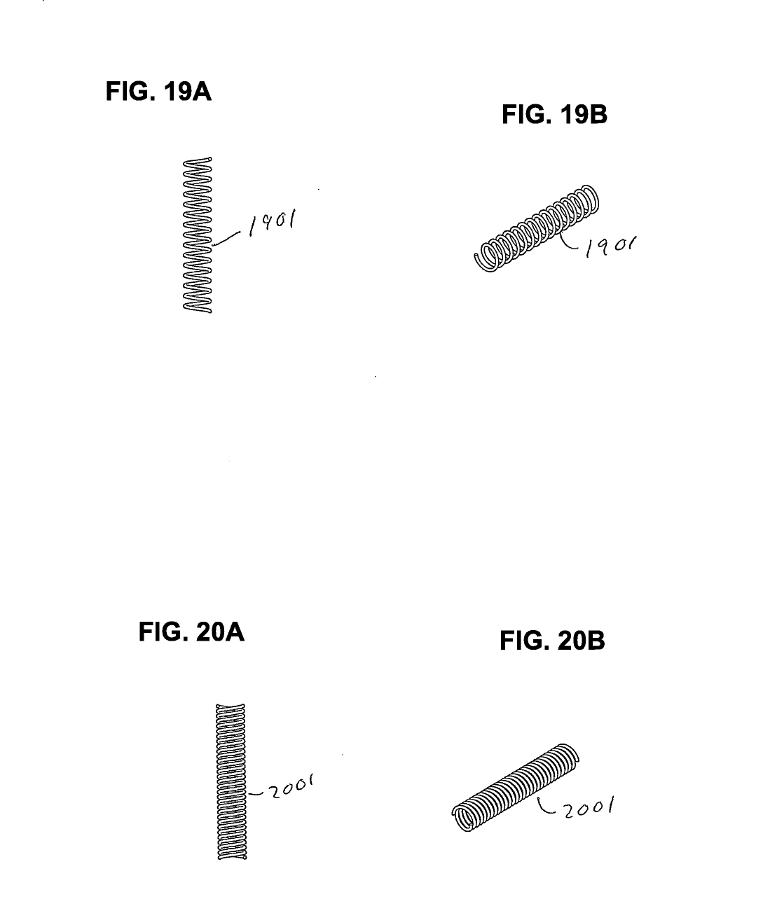

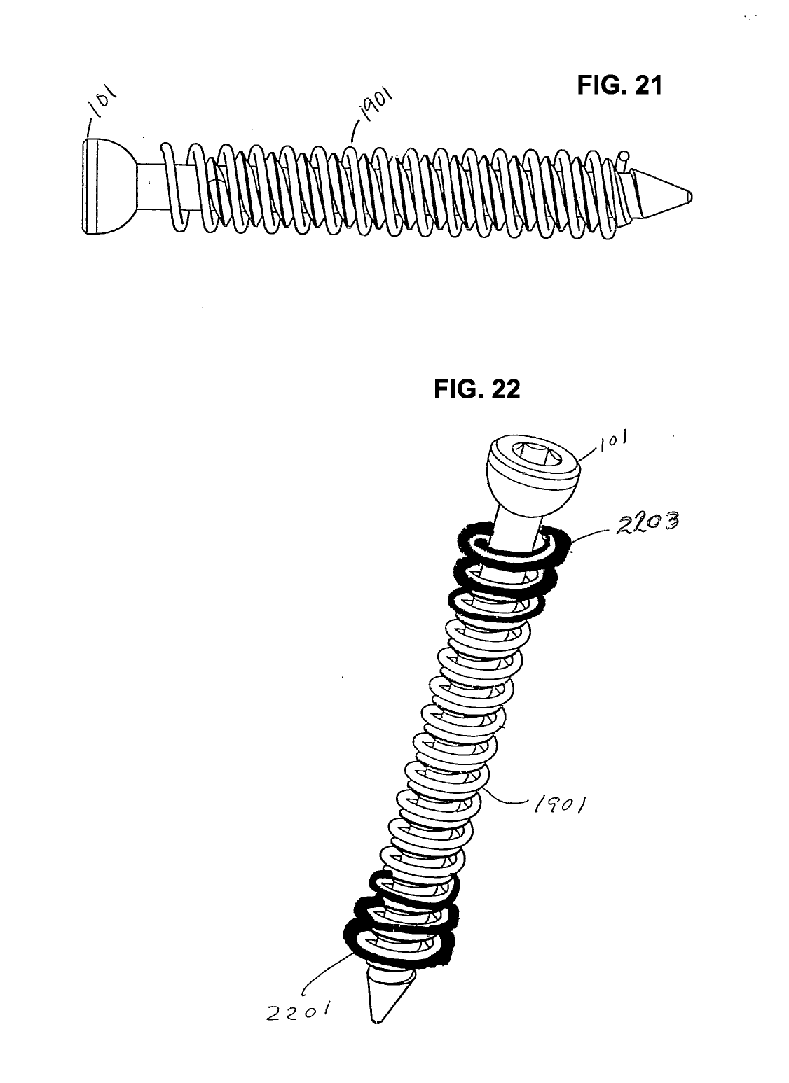

FIGS. 19a-b and 20a-b show variations of a helical fixation implant 1901 that contain no segments and wrap around a delivery device or fastener, such as, for example, 101 as shown in FIG. 21. FIG. 20 shows a tighter bound helical fixation implant 2001. FIG. 22 shows a helical fastener fixation implant 1901 where the thickness of the fixation implant is greater at the distal 2201 and proximal 2203 ends of the helical fastener fixation implant 1901.

### Fixation Implant Delivery Devices

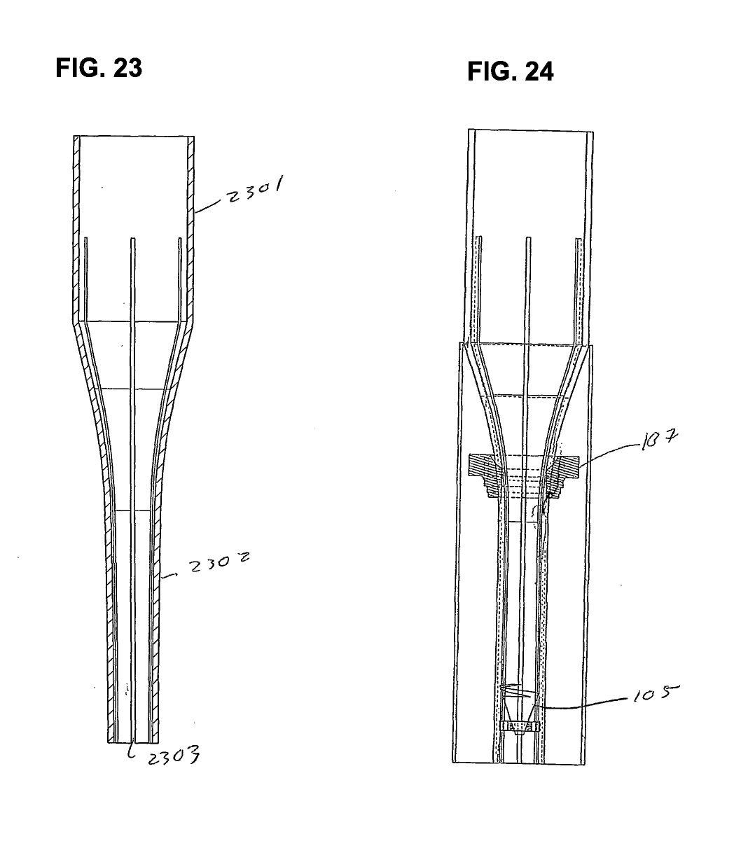

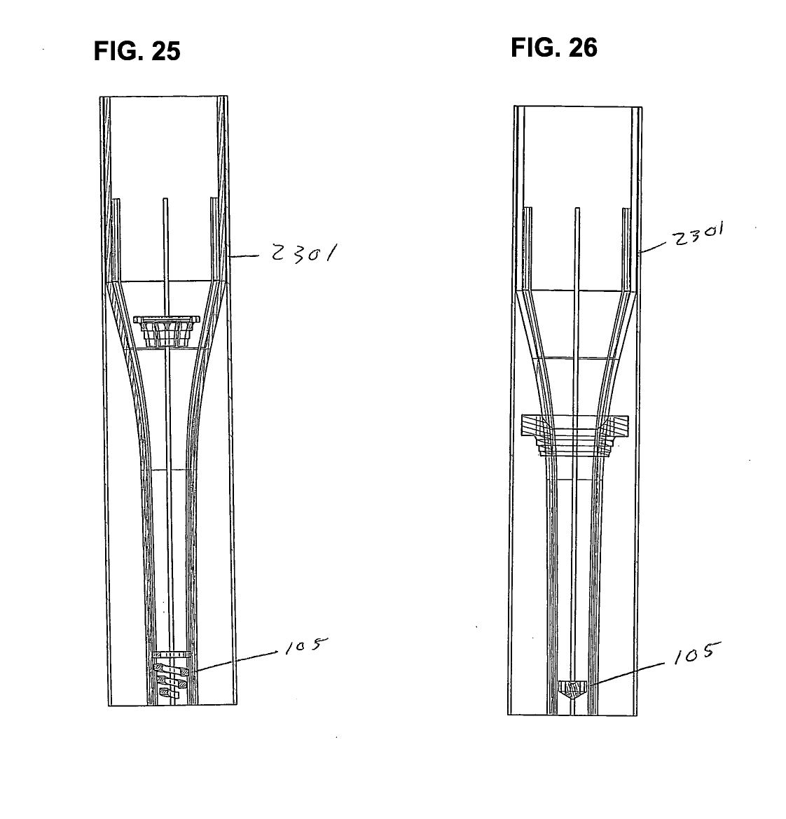

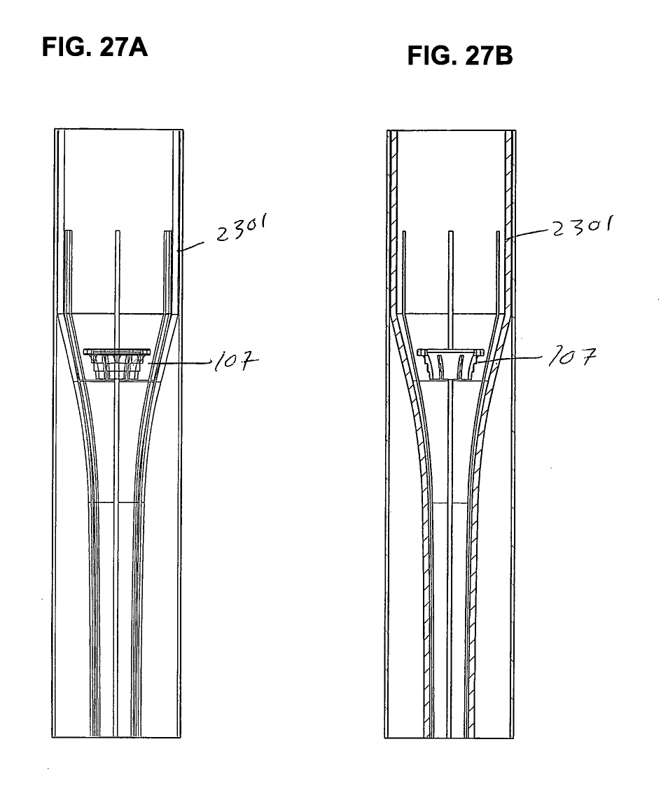

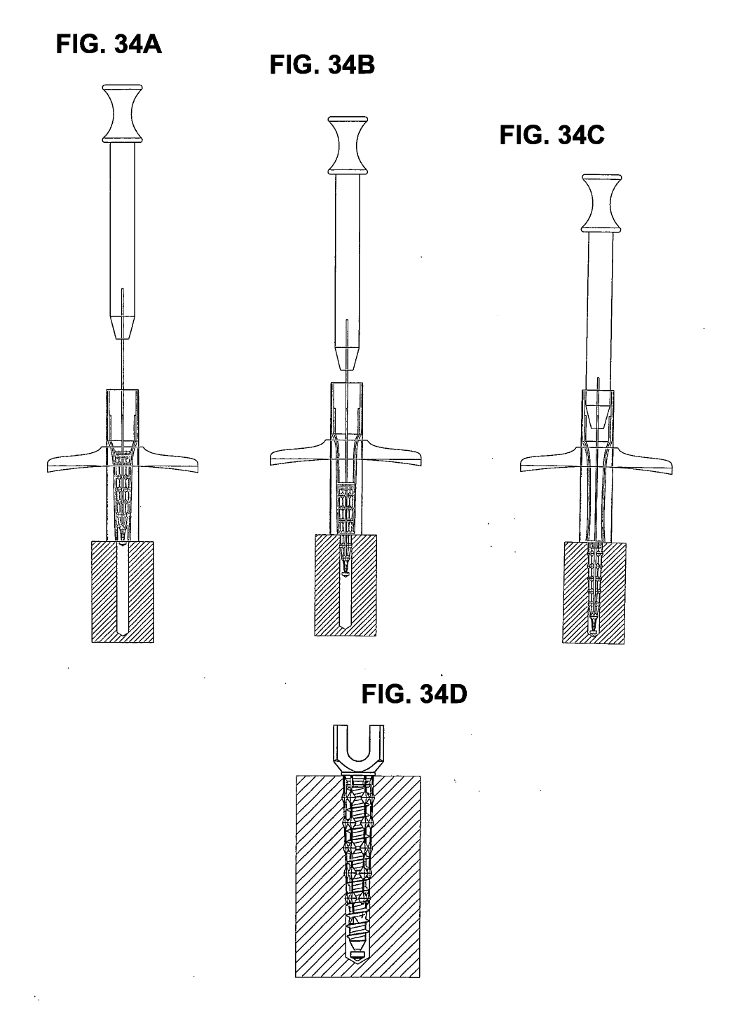

To facilitate the delivery of fastener fixation implants into a fastener opening in a controlled manner in order to achieve desired placement disclosed herein are various delivery components. In FIG. 23 a delivery portal cannula 2301 is shown. The delivery portal cannula 2301 consists of a cannula that narrows towards its distal end 2302. The distal end of the implant delivery portal cannula is inserted into the delivery device or fastener cavity space 2402. The delivery portal cannula 2301 has one or more slots 2303 that allow for expansion of the distal portion of the delivery portal cannula 2302 within the cavity space 2402. The fastener fixation implant with its implant guide rings is delivered into the cavity space through the delivery portal cannula 2301. FIGS. 24-26 show various fixation implant guide rings 105 and 107 located at the proximal and distal end of the portal. The segmented implants would be connected between the proximal 107 and distal 105 implant guide rings as shown in FIGS. 34a-d.

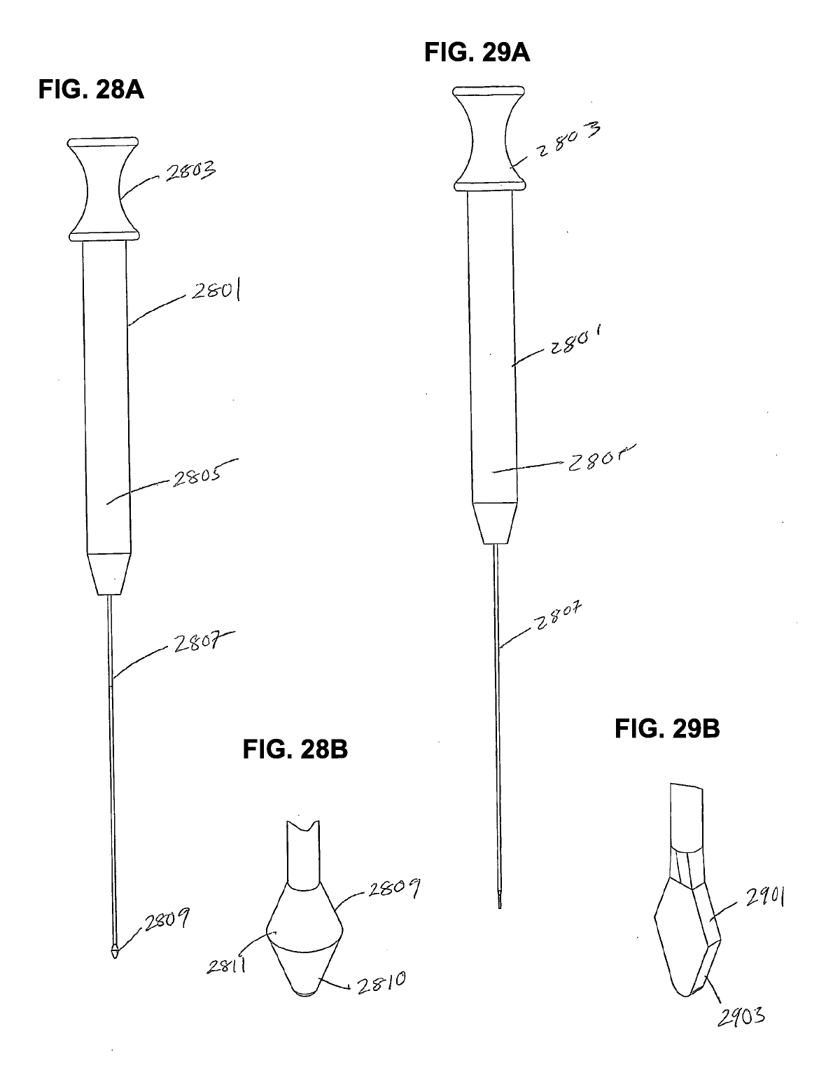

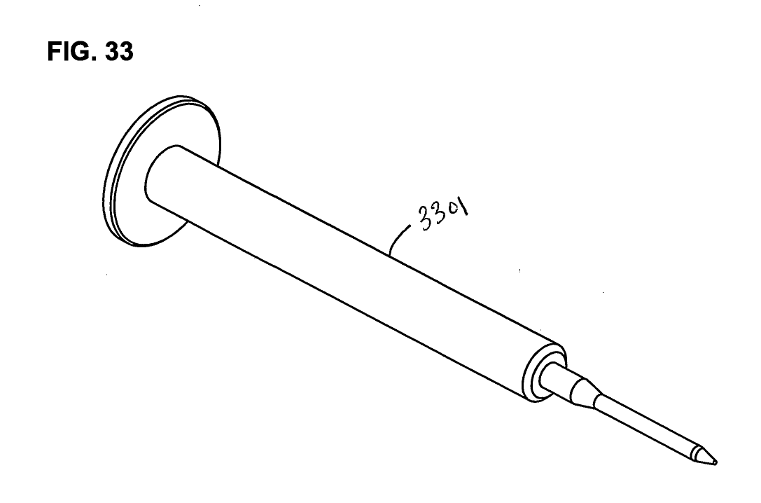

The implant guide rings are placed into the delivery portal cannula and delivered into the delivery device or fastener cavity with the use of a fixation implant delivery driver disclosed in FIGS. 28a-b. In the preferred embodiment the fixation implant delivery driver 2801 would consist of a handle grip 2803, a centralized body 2805, and narrow probe component 2807 and a driver tip 2809. In one embodiment the distal end 2810 of the driver tip 2809 is tapered to fit within a delivery portal cannula 2301. At the distal end of the driver tip 2807 the tip might be circumvental 2811 to allow for tamping of the placed fixation implant upon withdrawal after placements. In an alternative embodiment the delivery tip might be thin 2901 and tapered 2903 as shown in FIGS. 29a-b. FIG. 33 discloses a tamp 3301 that might be utilized after the guide rings and implants are delivered and placed in the desired location within the cavity. The tamp 3301 would facilitate the securing of the implant to the inner wall of the delivery device or fastener cavity.

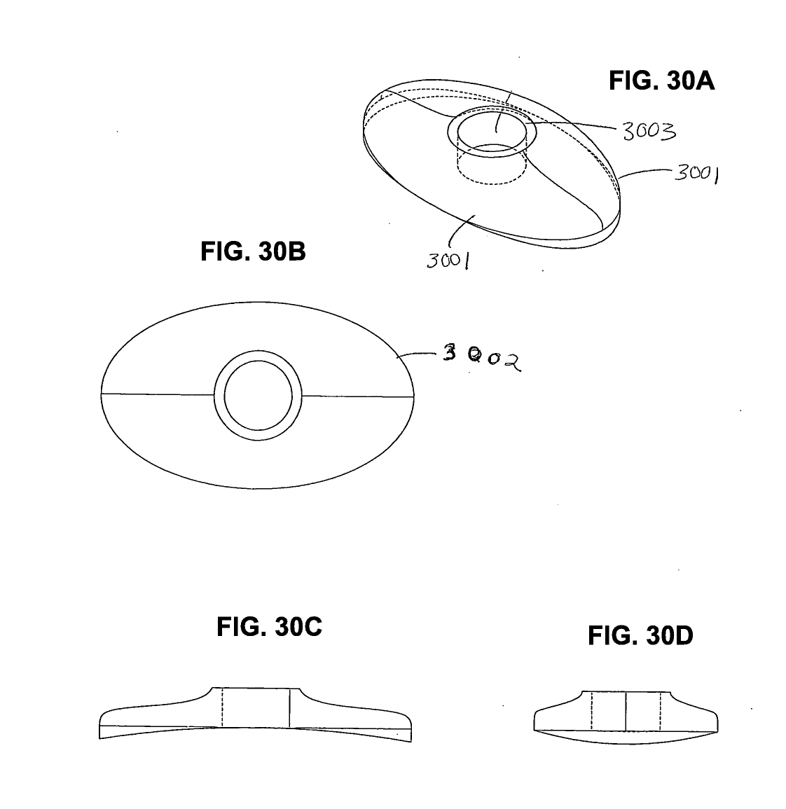

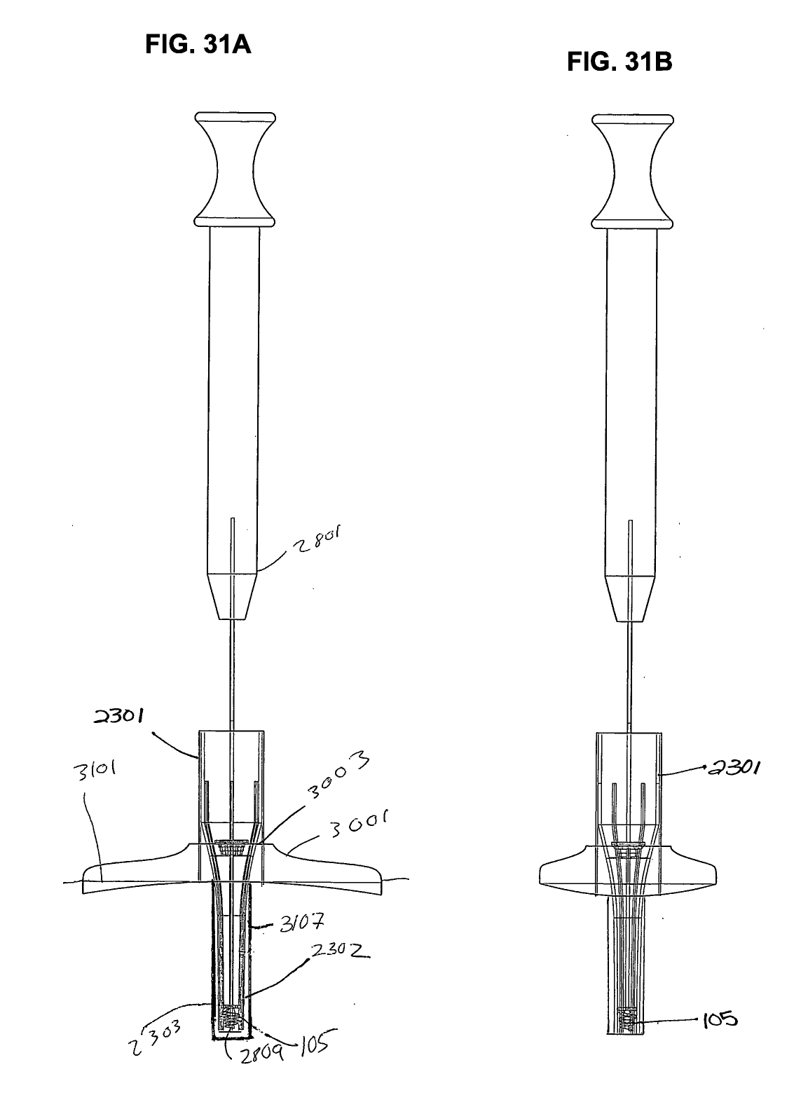

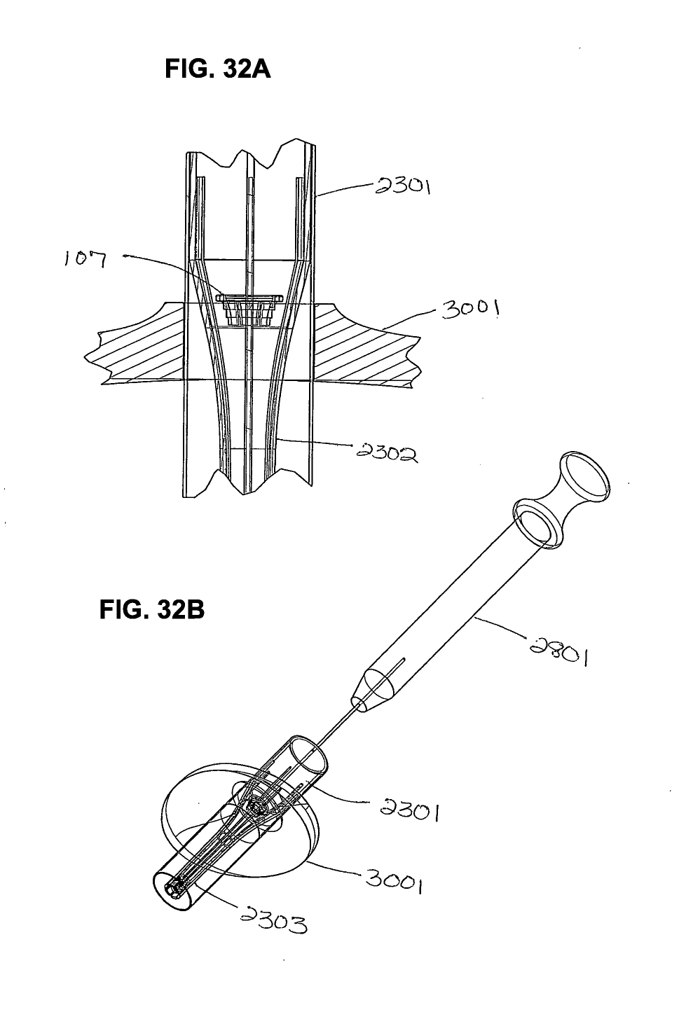

In one embodiment a delivery portal cannula stabilizer 3001 might be utilized as shown in FIGS. 30a-d and 31a-b. In this embodiment the stabilizer might be oblong 3002 and contain an access portal 3003 to allow for the delivery portal cannula 2301 and the fixation implant delivery device 2801. It could be of any shape that would provide stabilization of the delivery portal cannula. FIGS. 31a and b show the stabilizer 3001 placed on the outside of soft 3101 tissue with the delivery portal cannula 2301 and fixation implant delivery device 2801 passing through the access portal 3003. The distal end of the delivery portal cannula 2303 passes down into the fastener space 2302. The fixation implant delivery device tip 2809 then drives the distal implant guide ring 105 to the distal end of the fastener space 2302 placing the fixation implant 103 (not shown) in its desired location within the fastener space 2302. Upon withdrawal of the fixation implant delivery device 2801 the delivery tip 2809 disclosed in FIG. 28a—see might tamp the implant 103 segments into the inner substrate 3107 wall, such as bone wall of the fastener space, 2302 further securing the implant 103. FIG. 32 shows a different perspective of the delivery portal cannula and delivery device components.

It will be appreciated by persons skilled in the art that numerous variations and/or modifications may be made to the described device as specifically shown here without departing from the spirit or scope of that broader disclosure. The various examples are, therefore, to be considered in all respects as illustrative and not restrictive.

1-17. (canceled)

18. A fastener fixation apparatus securable in a substrate cavity, the apparatus comprising:
an elongate body having a distal end and a proximal end and being adapted to be disposed in the substrate cavity; and
a locking device comprising at least one implant comprising two or more rigidly connected segments;
wherein each rigidly connected segment is rigidly connected to each immediate adjacent segment by a rigid connecting member; and
wherein the two or more rigidly connected segments of the implant are in a linear array,
the locking device being adapted to, when inserted in the substrate cavity, become engaged against the elongate body to help secure the elongate body in the substrate cavity.

19. The apparatus of claim 18, wherein the at least one implant is attached to one or more rings disposed along the elongate body.

20. The apparatus of claim 19, wherein the at least one implant is attached to the one or more rings disposed along at the distal end of the elongate body.

21. The apparatus of claim 19, wherein the at least one implant is attached to the one or more rings disposed along the elongate body, at least one of the one or more rings is disposed at the distal end of the elongate body, and at least one of the one or more rings is disposed at the proximal end of the elongate body.

22. The apparatus of claim 35, wherein the elongate body comprises a fastener.

23. The apparatus of claim 22, wherein the fastener comprises a threaded elongate body.

24. The apparatus of claim 23, wherein the fastener comprises a bone screw.

25. The apparatus of claim 23, wherein the two or more rigidly connected segments of the implant are configured to couple with the threads of the fastener to secure the fastener within a non-soft body tissue cavity.

26. The apparatus of claim 35, wherein the connection material of the two or more rigidly connected segments of the implant is shaped to surround a fastener so as to be fixated in position along the fastener.

27. The apparatus of claim 35, wherein the locking device comprising at least one implant is comprised of three or more rigidly connected segments;

28. The apparatus of claim 27, wherein a material connecting the three or more rigidly connected segments of the implant is shaped in a zigzag configuration.

29. The apparatus of claim 28, wherein the material connecting the three or more rigidly connected segments of the implant shaped in a zigzag configuration is criss crossed to form a criss crossed zigzag shaped implant.

30. The apparatus of claim 29, wherein two or more of the criss crossed zigzag shaped implants are intertwined with each other.

31. The apparatus of claim 30, wherein the two or more of the intertwined criss crossed zigzag shaped implants surround a fastener.

32. The apparatus of claim 27, wherein the connection material of the three or more rigidly connected segments of the implant is shaped in a sinusoidal wave configuration.

33. A fastener fixation apparatus securable in a substrate cavity, the apparatus comprising:
an elongate body having a distal end and a proximal end and being adapted to be disposed in a substrate cavity; and
a locking device comprising at least one rigid helical shaped implant.
the locking device being adapted to, when inserted in the substrate cavity, become engaged against the elongate body to help secure the elongate body in the substrate cavity.

34. The apparatus of claim 33, wherein the rigid helical shaped implant is attached to one or more rings disposed along the elongate body.

35. The apparatus of claim 33, wherein the thickness of rigid helical shaped implant is thicker at one end of the rigid helical shaped implant.

36. The apparatus of claim 33, wherein the thickness of the rigid helical shaped implant is thicker at both the proximal and the distal end of the rigid helical shaped implant.

37. The apparatus of claim 33, wherein the elongate body comprises a fastener.
